(19) United States
(12) Patent Application Publication
(10) Pub. No.: US 2022/0211767 A1
(43) Pub. Date: Jul. 7, 2022

(54) ENHANCEMENT OF FIBROBLAST THERAPEUTIC ACTIVITY BY T CELL MODULATION

(71) Applicant: Figene, LLC, Houston, TX (US)

(72) Inventors: Pete O'Heeron, Houston, TX (US); Thomas Ichim, San Diego, CA (US)

(21) Appl. No.: 17/594,730

(22) PCT Filed: Apr. 27, 2020
(86) PCT No.: PCT/US2020/030045

§ 371 Date: Oct. 27, 2021

Related U.S. Application Data
(60) Provisional application No. 62/839,652, filed on Apr. 27, 2019.

(51) Int. Cl. A61K 35/33; A61K 38/20; A61K 39/395; A61K 38/57

(52) U.S. Cl. CPC A61K 35/33; A61K 38/57; A61K 39/3955; A61K 38/2013

(57) ABSTRACT

Embodiments of the disclosure concern methods and compositions for enhancing therapeutic efficacy of fibroblast therapy for an individual in need thereof. The disclosure concerns administering to the individual one or both of interleukin-2 (IL-2) and one or more anti-CD3 agents to enhance efficacy of the fibroblast therapies. In specific cases, the IL-2 is provided to the individual at particular low doses, and the anti-CD3 agent may be a monoclonal antibody.

This application claims priority to U.S. Provisional Patent Application Ser. No. 62/839,652, filed Apr. 27, 2019, which is incorporated by reference herein in its entirety.

### TECHNICAL FIELD

Embodiments of the disclosure encompass at least the fields of cell biology, molecular biology, cell therapy, immunology, and medicine.

### BACKGROUND

The immune systems of healthy individuals are tolerant to the body's own self-antigens. Tolerance is a state of immunological unresponsiveness to an antigen, and autoimmunity occurs when tolerance is not present for a self-antigen. Therapies for autoimmune diseases have been hampered because the cause of autoimmunity is often multifactorial with complicated etiologies involving multiple autoantigens as targets. Because of the complicated nature of the immune system, prior therapies attempt to achieve a general suppression of the immune system when treatment of transplant rejection or autoimmunity was desired. Unfortunately, currently used immune suppressive agents prevent all T cell responses by depletion or inactivation of T cells. This is seen, as an example, in the use of glucocorticoids and the calcineurin inhibitors, such as cyclosporine A and FK-506, which block cytokine gene transcription, preventing the production of T cell growth factors. Other immune suppressive agents, such as Campath 1H, cause prolonged depletion of T cells. While these approaches are effective in the short term, their effects are not antigen-specific and may not persist after the drugs are discontinued. Hence, true immunologic tolerance, in which an immune response does not occur after an immune suppressive agent is withdrawn, is rarely achieved.

The disclosure encompasses the use of immune modulation to induce acceptance of allogeneic fibroblast cell populations possessing therapeutic activities, as well as utilization of immune modulation to augment enhanced therapeutic activity.

### BRIEF SUMMARY

Embodiments of the disclosure pertain to the field of cellular therapy, more particularly the disclosure concerns the field of utilizing fibroblasts for a therapeutic purpose, for example to elicit regenerative activities in an individual in need thereof. More specifically, the disclosure encompasses methods that overcome some of the limitations surrounding the use of allogeneic fibroblasts by utilizing clinically-tested pharmacological drugs to modulate the immune system in order to accept foreign fibroblasts, as well as to augment therapeutic activity of the fibroblasts.

In specific embodiments, the methods and compositions of the disclosure utilize one or more particular compounds to increase efficacy of fibroblasts transplanted from one person to another. In specific cases, one of the compounds is interleukin (IL)-2, including at a low dose, and another is an anti-CD3 agent, such as an anti-CD3 antibody. In particular embodiments, one or both independently trigger production of T regulatory cells that enhance fibroblast regenerative activity and also stop allogeneic fibroblasts from being rejected in a recipient individual.

In one embodiment, there is a method of providing a fibroblast therapy to an individual in need thereof, comprising the step of providing to the individual an effective amount of the fibroblast therapy and one or both of IL-2 and one or more anti-CD3 agents. The fibroblast therapy may be autologous, allogeneic, or xenogeneic with respect to the individual. The order of delivery of the different compositions may be of any kind.

In some embodiments, the (1) fibroblast therapy, (2) IL-2, and (3) anti-CD3 agent(s) are provided to the individual in any order, including in the order of (1), (2), (3); (1), (3), (2); (2), (1), (3); (2), (3), (1); (3), (1), (2); or (3), (2), (1). In some cases, two or all of (1), (2), and (3) are provided to the individual simultaneously, such as (1) and (2) simultaneously; (1) and (3) simultaneously; (2) and (3) simultaneously; or (1), (2) and (3) simultaneously. When one or more of (1), (2), and (3) are provided to the individual at different times, the duration between deliveries may be of any duration, including a duration of 1-60 minutes, 1-24 hours, 1-7 days, 1-4 weeks, 1-12 months, and any subrange therebetween. When two or more of (1), (2), and (3) are provided to an individual at the same, time, they may or may not be provided to the individual in the same formulation or composition. In one specific embodiment, (2) and (3) are provided to the individual prior to (1).

In at least some cases, the fibroblasts are plastic-adherent and in any case the fibroblasts may be derived from a tissue selected from the group consisting of a) adipose; b) omentum; c) subintestinal mucosa; d) placenta; e) cord blood; f) wharton's jelly; g) bone marrow; h) peripheral blood; i) hair follicle; j) skin; k) cutis; l) tonsil; m) peripheral blood; n) menstrual blood; o) thymus; and p) a combination thereof. The fibroblasts may express one or more certain markers and/or lack expression of one or more certain markers. In some cases, the fibroblasts express one or more markers selected from the group consisting of a) CD73; b) CD56; c) CD140; d) CD105; e) CD90; and f) a combination thereof. The fibroblasts may possess a younger biological age than the individual. The fibroblasts may be modified, such as transfected with one or more recombinant nucleic acids. In some cases, the fibroblasts are transfected with hTERT and/or one or more of Oct-4, NANOG, and SOX-2. The fibroblasts may or may not be dedifferentiated. The fibroblasts may be exposed to, transfected with, or both of one or more DNA methyltransferase inhibitors, one or more histone deacetylase inhibitors, one or more inhibitors of GSK-3, or a combination thereof. The fibroblasts may be transfected with cytoplasm from cells younger than the fibroblasts, including stem cells, such as pluripotent stem cells, for example.

In some embodiments, the IL-2 is administered to the individual at a concentration of 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0×10⁶ IU/m²/day. The IL-2 may be administered to the individual at a dose between 0.3×10⁶ IU/m²/day and 3.0×10⁶ IU/m²/day. The IL-2 may be administered to the individual at a dose no more than 20.0×10⁶ IU/m²/day. The IL-2 may be administered to the individual daily. The IL-2 may be administered to the individual for 1-16 weeks, including daily to the individual for 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 1-11, 2-11, 3-11, 4-11, 5-11, 6-11, 7-11, 8-11, 9-11, 10-11, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4- 6, 5-6, 1-5, 2-5, 3-5, 4-5, 1-4, 2-4, 3-4, 1-3, 2-3, or 1-2 weeks. The IL-2 may be administered as a continuous infusion and/or by subcutaneous injection. The IL-2 may or may not be administered prior to the fibroblasts. In some cases, the IL-2 is administered prior to the fibroblasts in a range of about 1 hour prior to about 3 weeks prior. The IL-2 may administered about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 3 days, 4 days, 5, days, 6 days, or 1 week prior to the fibroblasts.

In some cases, the anti-CD3 agent is an antibody, aptamer, siRNA or shRNA, or combination thereof. In cases where the anti-CD3 agent is an antibody, it may be an antibody of any kind, including a monoclonal antibody. The individual may be provided the anti-CD3 agent prior to delivery of the fibroblasts. The anti-CD3 agent may or may not be provided to the individual 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, and/or 1 day before administration of the fibroblasts to the individual. The individual may also be provided an effective amount of one or more tolerance inducing agents, such as alpha1-antitrypsin.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the disclosure may “consist essentially of” or “consist of” one or more sequences of the invention, for example. Some embodiments may consist of or consist essentially of one or more elements, method steps, and/or methods of the invention. It is contemplated that any method or composition described herein can be implemented with respect to any other method or composition described herein. The scope of the present application is not intended to be limited to the particular embodiments of the process, machine, manufacture, composition of matter, means, methods and steps described in the specification.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe etc. A second exemplary method of administering is by a direct mechanism such as, local tissue administration, oral ingestion, transdermal patch, topical, inhalation, suppository etc.

As used herein “allogeneic” refers to tissues or cells from another body that in a natural setting are immunologically incompatible or capable of being immunologically incompatible, although from one or more individuals of the same species in some cases.

The term “individual” or “subject” that may be used interchangeably, as used herein, refers to a human or animal that may or may not be housed in a medical facility and may be treated as an outpatient of a medical facility. The individual may or may not be receiving one or more medical compositions from a medical practitioner and/or via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method and/or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

The term “pharmaceutically” or “pharmacologically acceptable,” as used herein, refer to molecular entities and compositions that do not produce adverse, allergic, or other untoward reactions when administered to an animal or a human.

As used herein, the term “therapeutically effective amount” is synonymous with “effective amount,” “therapeutically effective dose,” and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells.

As used herein, the term “transplantation” refers to the process of taking living tissue and/or cells and implanting it in another part of the body or into another body.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

Disclosed are methods of enhancing angiogenic and regenerative activity of fibroblast cells through enhancement of interaction with T regulatory cells. In one embodiment, fibroblasts are administered together with one or more agents capable of increasing number and/or activity of T cells possessing angiogenic activity, although in some cases the fibroblasts are provided to the individual at a time different from the administration of the one or more agents.

In one embodiment, fibroblasts are administered together with low dose IL-2 at a concentration capable of augmenting populations of CD4 T cells that possess the ability to stimulate angiogenesis. Furthermore, IL-2 may be administered at a low dose to enhance engraftment and generate augmented functionality and viability of allogeneic fibroblasts administered in a therapeutic setting. In another embodiment, fibroblasts are administered together with anti-CD3 agent, such as an anti-CD3 monoclonal antibody, at a concentration and frequency sufficient to enhance fibroblast therapeutic activity through induction of tolerogenesis as well as stimulation of angiogenic activity. In some cases the fibroblasts are administered with both IL-2 and the anti-CD3 antibody, although the different compounds may be delivered at different times.

In some embodiments of the disclosure, IL-2 is administered as Aldesleukin (Proleukin, Novartis), which is a commercially available IL-2 licensed for the treatment of metastatic renal cell carcinoma in the UK. It is produced by recombinant DNA technology using an Escherichia coli strain, which contains a genetically engineered modification of the human IL-2 gene, and is administered either intravenously or subcutaneously (SC), as examples. Following short intravenous infusion, its pharmacokinetic profile is typified by high plasma concentrations, rapid distribution into the extravascular space and a rapid renal clearance. The recommended doses for continuous infusion and subcutaneous injection (as detailed in the Summary of Product Characteristics) are repeated cycles of 18×10⁶ IU per m² per 24 hours for 5 days and repeated doses of 18×10⁶ IU, respectively. Peak plasma levels are reached in 2-6 hours after SC administration, with bioavailability of Aldesleukin ranging between 31% and 47%. The process of absorption and elimination of subcutaneous Aldesleukin is described by a one-compartment model, with a 45 min absorption half-life and an elimination half-life of 3-5 hours W. Any IL-2 used in methods of the disclosure may be administered as directed for any commercially available IL-2.

Natural IL-2 was first identified in 1976 as a growth factor for T lymphocytes. It is produced by human cluster designation (CD) 4+ T-cells and some CD8+ T-cells and is synthesized mainly by activated T-cells, in particular CD4⁺ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilitates the proliferation and differentiation of B-cells and the synthesis of immunoglobulin by B-cells, and stimulates the generation, proliferation and activation of natural killer (NK). IL-2 is known to play a central role in the generation of immune responses. In cancer clinical trials, high-dose recombinant IL-2 (e.g., IV bolus dose of 600,000 international units (IU)/kg every 8 hours for up to 14 doses) demonstrated antitumor activity in metastatic renal cell carcinoma (RCC) and metastatic melanoma (and the present disclosure contemplates use of doses less than this). Accordingly, such high-dose IL-2 was approved for the treatment of metastatic RCC in Europe in 1989 and in the US in 1992. In 1998, approval was obtained to treat patients with metastatic melanoma. Recombinant human IL-2 (Aldesleukin) (Proleukin®-Novartis Inc. & Prometheus Labs Inc.) is currently approved by the United States Food and Drug Administration (US FDA). However, IL-2 has a dual function in the immune response in that it not only mediates expansion and activity of effector cells, but also is crucially involved in maintaining peripheral immune tolerance. A major mechanism underlying peripheral self-tolerance is IL-2-induced activation-induced cell death (AICD) in T cells. AICD is a process by which fully activated T cells undergo programmed cell death through engagement of cell surface-expressed death receptors such as CD95 (also known as Fas) or the TNF receptor. When antigen-activated T cells expressing a high-affinity IL-2 receptor (after previous exposure to IL-2) during proliferation are re-stimulated with antigen via the T cell receptor (TCR)/CD 3 complex, the expression of Fas ligand (FasL) and/or tumor necrosis factor (TNF) is induced, making the cells susceptible for Fas-mediated apoptosis. This process is IL-2 dependent and mediated via STATS. By the process of AICD in T lymphocytes, tolerance can not only be established to self-antigens, but also to persistent antigens that are clearly not part of the host's makeup, such as tumor antigens.

In one embodiment of the disclosure, patients receiving fibroblast therapy are pretreated with 0.3×10⁶ IU of IL-2 (such as aldesleukin), such as daily. Concentrations for clinical uses of aldesleukin (as one example) could be used from the literature as described for other indications including heart failure [1], Wiskott-Aldrich syndrome [2], Graft Versus Host Disease [3, 4], lupus [5], type 1 diabetes [6-8] and are incorporated by reference. In some embodiments of the disclosure, administration occurs of low doses of IL-2 in the form of aldesleukin (as one example) every day at concentrations of 0.3×10⁶ to 3.0×10⁶ IU IL-2 per square meter of body surface area for 8 weeks, or in other embodiments repetitive 5-day courses of 1.0×10⁶ to 3.0×10⁶ IU IL-2. Various types of IL-2 may be utilized. Examples of IL-2 variants, recombinant IL-2, methods of IL-2 production, methods of IL-2 purification, methods of formulation, and the like are well known in the art and can be found, for example, at least in U.S. Pat. Nos. 4,530,787, 4,569,790, 4,572,798, 4,604,377, 4,748,234, 4,853,332, 4,959,314, 5,464,939, 5,229,109, 7,514,073, and 7,569,215, each of which is herein incorporated by reference in their entirety for all purposes.

In some embodiments of the disclosure, fibroblasts are administered together with one or more tolerance-inducing agents (that may also be referred to as a tolerance-restoring agents), and said “agent” is meant to encompass essentially any type of molecule that can be used to impart one or more therapeutic properties to fibroblasts administered in an allogeneic host. Proteins, such as antibodies, fusion proteins, and soluble ligands, any of which may either be identical to a wild-type protein or contain a mutation (i.e., a deletion, addition, or substitution of one or more amino acid residues), and the nucleic acid molecules that encode them (or that are “antisense” to them; e.g., an oligonucleotide that is antisense to the nucleic acids that encode a target polypeptide, or a component (e.g., a subunit) of their receptors), are all “agents.” These agents of the disclosure can either be administered systemically, locally, or by way of cell-based therapies (i.e., an agent of the disclosure can be administered to a patient by administering a cell that expresses that agent to the patient). As one example, a tolerance-restoring agent can be alpha1-antitrypsin (AAT; sometimes abbreviated A1AT), which is also referred to as alpha1-proteinase inhibitor. AAT is a major serum serine-protease inhibitor that inhibits the enzymatic activity of numerous serine proteases including neutrophil elastase, cathespin G, proteinase 3, thrombin, trypsin and chymotrypsin. For example, one can administer an AAT polypeptide (e.g., a purified or recombinant AAT, such as human AAT) or a homolog, biologically active fragment, or other active mutant thereof. Alpha1 proteinase inhibitors are commercially available for the treatment of AAT deficiencies, and include ARALAST™, PROLASTIN™ and ZEMAIRA™. The AAT polypeptide or the biologically active fragment or mutant thereof can be of human origin and can be purified from human tissue or plasma. Alternatively, it can be recombinantly produced. It is to be understood that, whenever a full-length, naturally occurring AAT can be used, a biologically active fragment or other biologically active mutant thereof (e.g., a mutant in which one or more amino acid residues have be substituted) can also be used. A naturally occurring polypeptide (e.g., AAT) can be purified from a natural source or recombinantly produced, as both forms may be useful. Similarly, any polypeptide can be of human or non-human origin. While there may be advantages to administering a human protein, the methods and compositions of the disclosure are not so limited.

The methods of the present disclosure (e.g., multiple-variable dose IL-2 alone or in combination with one or more other anti-immune disorder therapies) can be administered to a desired subject or once a subject is indicated as being a likely responder to such therapy. In another embodiment, the therapeutic methods of the present disclosure can be avoided if a subject is indicated as not being a likely responder to the therapy and an alternative treatment regimen, such as targeted and/or untargeted anti-immune therapies, can be administered.

In one embodiment, a multiple-variable IL-2 dose method of treating a subject receiving fibroblast therapy comprises a) administering to the subject an induction regimen comprising continuously administering to the subject IL-2 at a dose that increases the subject's plasma IL-2 level and increases the subject's ratio of immune suppressive T cells to conventional T lymphocytes (Tcons) and b) subsequently administering to the subject at least one maintenance regimen comprising continuously administering to the subject an IL-2 maintenance dose that is higher than the induction regimen dose (an example of a maintenance does is 4-20×10⁶ IU/m²/day) and that i) further increases the subject's plasma IL-2 level and ii) further increases the ratio of immune suppressive T cells to Tcons, thereby treating the subject, is provided. In one embodiment, the level of plasma IL-2 resulting from the induction regimen is depleted below that of the prior peak plasma IL-2 level before the induction regimen. The IL-2 maintenance regimen can, in certain embodiments, increase the subject's plasma IL-2 level beyond the peak plasma IL-2 level induced by the induction regimen. The term “multiple-variable IL-2 dose method” refers to a therapeutic intervention comprising more than one IL-2 administration, wherein the more than one IL-2 administration uses more than one IL-2 dose. Such a method is contrasted from a “fixed” dosed method wherein a fixed amount of IL-2 is administered in a scheduled manner, such as daily. The term “induction regimen” refers to the continuous administration of IL-2 at a dose that increases the subject's plasma IL-2 level and increases the subject's immune suppressive T cells:Tcons ratio. In some embodiments, the regimen occurs until a peak level of plasma IL-2 is achieved. The subject's plasma IL-2 level and/or immune suppressive T cell:Tcons ratio can be increased by at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200% or more relative to the baseline ratio prior to initiation of therapy.

In one embodiment of the disclosure, certain doses and methods according to FDA-approved uses, Tcons are activated relative to immune suppressive T cells such that the immune suppressive T cells:Tcons ratio actually decreases. By contrast, the methods of the present disclosure increase the immune suppressive T cells:Tcons ratio by using “low-dose IL-2” in a range determined herein to particularly promote immune suppressive T cells over Tcons and that are safe and efficacious in subjects having received fibroblasts.

The term “low-dose IL-2” refers to the dosage range wherein immune suppressive T cells are enhanced relative to Tcons. In one embodiment, low-dose IL-2 refers to IL-2 doses that are less than or equal to 50% of the “high-dose IL-2” doses (e.g., 18 million IU per m² per day to 20 million IU per m² per day, or more) used for anti-cancer immunotherapy. The upper limit of “low-dose IL-2” can further be limited by treatment adverse events, such as fever, chills, asthenia, and/or fatigue. IL-2 is generally dosed according to an amount measured in international units (IU) administered in comparison to body surface area (BSA) per given time unit. BSA can be calculated by direct measurement or by any number of well-known methods (e.g., the Dubois & Dubois formula). Generally, IL-2 is administered according in terms of IU per m² of BSA per day. Exemplary low-dose IL-2 doses according to the methods of the present disclosure include, in terms of 10⁶ IU/m²/day, any one of 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0×10⁶ IU/m²/day, including any values in between and/or ranges in between. For example, an induction regimen dose can range between 0.3×10⁶IU/m²/day and 3.0×10⁶ IU/m²/day with any value or range in between.

The term “continuous administration” refers to administration of IL-2 at regular intervals without any intermittent breaks in between. Thus, no interruptions in IL-2 occur. For example, the induction dose can be administered every day (e.g., once or more per day) during at least 1-14 consecutive days or any range in between (e.g., at least 4-7 consecutive days). As described herein, longer acting IL-2 agents and/or IL-2 agents administered by routes other than subcutaneous administration are contemplated. Intermittent intravenous administration of IL-2 described in the art results in short IL-2 half-lives incompatible with increasing plasma IL-2 levels and increasing the immune suppressive T cells:Tcons ratio according to the present disclosure. However, once-daily subcutaneous IL-2 dosing, continuous IV infusion, long-acting subcutaneous IL-2 formulations, and the like are contemplated for achieving a persistent steady state IL-2 level.

IL-2 (and any other agent or composition encompassed herein) can be administered in any pharmaceutically acceptable formulation and by any suitable administration route, such as by subcutaneous, intravenous, intraperitoneal, oral, nasal, transdermal, or intramuscular administration. In one embodiment, the present disclosure provides pharmaceutically acceptable compositions that comprise IL-2 at a therapeutically effective amount, formulated together with one or more pharmaceutically acceptable carriers (additives) and/or diluents. The pharmaceutical compositions of the present disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

In some embodiments of the disclosure, one or more anti-CD3 agents are provided to the individual, such as an antibody (including monoclonal), aptamer, shRNA, siRNA, and so forth. In specific embodiments, a monoclonal antibody (mAb) against the CD3 molecule is utilized, including for immune modulation. This approach has previously been used to induced tolerance to autoimmunity in murine models of type 1 diabetes mellitus. Treatment with anti-CD3 mAb reversed diabetes in the NOD mouse and prevented recurrent immune responses toward transplanted syngeneic islets. This was achieved without the need for continuous immune suppression and persisted at a time when T cell numbers were not depleted and were quantitatively normal. Another approach is to induce specific immunological unresponsiveness by administering self-antigens.

For the practice of the disclosure, in specific embodiments one utilizes a particular type of anti-CD3 antibody. The anti-CD3 antibody may be of any kind, including polyclonal, monoclonal, bispecific, as a heteroconjugate antibody, a humanized antibody, and so forth. The natural role of CD3 is to transduce signals in T cells from the T cell receptor into the nucleus of the T cells, usually to activity T cells. In some situations, antibodies to CD3 cause activation of T cells, not suppression. For example, Hirsch et al. investigated the ability of low dose anti-CD3 to enhance an anti-tumor response directed against the malignant murine UV-induced skin tumor. Low dose anti-CD3 administration resulted in enhanced in vitro anti-tumor activity and prevented tumor outgrowth in approximately two-thirds of animals treated at the time of tumor inoculation. Furthermore, these animals displayed lasting tumor-specific immunity. Augmentation of various parameters of immunity was noted. These results suggested that anti-CD3 mAb can be utilized for the enhancement of anti-tumor responses in vivo and may have general application in the treatment of immunodeficiency. They also point to the care that needs to be exercised when manipulating the CD3 pathway, given that the pathway can be both capable of upregulating or inhibitory [9]. Activatory signals by crosslinking CD3 are also seen in the tumor infiltrating lymphocyte (TIL) culture systems. It is known that early in the life of the TIL bulk culture, cytotoxicity is non-major histocompatibility complex restricted. Under these culture conditions antitumor cytotoxicity was observed to decline with increasing age of the bulk culture. In addition, TIL became refractory to IL-2-induced expansion. In one study, scientists have used solid-phase anti-CD3 antibodies for TIL activation followed by culture in reduced concentrations of IL-2 to reactivate TIL previously grown in high concentrations of rIL-2. TIL refractory to IL-2 in terms of growth and antitumor cytotoxicity proved sensitive to anti-CD3 activation. The use of solid-phase anti-CD3 was also more effective than high concentrations of IL-2 in the expansion of TIL when used at the start of culture. Finally, TIL could be induced to secrete IL-2 following solid-phase activation with anti-CD3. These data suggest that human TIL are susceptible to activation by signals directed at the CD3 complex of the TIL cell surface [10].

An example of how different CD3 targeting antibodies can elicit different effects is seen in another study, which Davis et al. examined the IgM monoclonal antibody called 38.1, which was distinct from other anti-CD3 mAb, in that it was rapidly modulated from the cell surface in the absence of a secondary antibody. Although 38.1 induced an immediate increase in intracellular free calcium [Ca2+]i by highly purified T cells, it did not induce entry of the cells into the cell cycle in the absence of accessory cells (AC) or a protein kinase C-activating phorbol ester. Treated T cells were markedly inhibited in their capacity to respond to the T cell stimulating mitogen phytohemagluttanin. Inhibition of responsiveness could be overcome by culturing the cells with supplemental antigen presenting cells or the cytokine IL-2. These studies demonstrate that a state of T cell nonresponsiveness can be induced by modulating CD3 with an anti-CD3 mAb in the absence of co-stimulatory signals. A brief increase in [Ca2+]i resulting from mobilization of internal calcium stores appears to be sufficient to induce this state of T cell nonresponsiveness [11].

In some situations, anti-CD3 antibodies have been shown to program T cells towards antigen-specific tolerance. This is illustrated in one example in the work of Anasetti et al. who exposed PBMC to alloantigen for 3-8 days in the presence of anti-CD3 antibodies. They showed no response after restimulation with cells from the original donor but the PBMC remained capable of responding to third-party donors. Antigen-specific nonresponsiveness was induced by both nonmitogenic and mitogenic anti-CD3 antibodies but not by antibodies against CD2, CD4, CD5, CD8, CD18, or CD28. This suggested the unique ability of this protein to modulate programs in the T cells that are antigen specific. Nonresponsiveness induced by anti-CD3 antibody in mixed leukocyte culture was sustained for at least 34 d from initiation of the culture and 26 d after removal of the antibody. Anti-CD3 antibody also induced antigen-specific nonresponsiveness in cytotoxic T cell generation assays. Anti-CD3 antibody did not induce nonresponsiveness in previously primed cells [12].

In specific embodiments, the use of anti-CD3 antibodies for the practice of the disclosure considers that the antibodies not only do not result in activation of T cell proliferation and inflammatory cytokine secretion, but also that the T cells actually inhibit inflammation and promote regeneration.

In one embodiment of the disclosure, anti-CD3 antibody is given at a certain time period prior to administration of the fibroblasts. In specific embodiments the administration of the anti-CD3 antibody occurs 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, and/or 1 day before administration of fibroblasts. In one specific example, a 14-day course of the anti-CD3 monoclonal antibody utilizes the antibody hOKT3γ1 (Ala-Ala) administered intravenously (1.42 μg per kilogram of body weight on day 1; 5.67 μg per kilogram on day 2; 11.3 μg per kilogram on day 3; 22.6 μg per kilogram on day 4; and 45.4 μg per kilogram on days 5 through 14); these doses were based on those previously used for treatment of transplant rejection [13] which is incorporated by reference. Other types of anti-CD3 molecules and dosing regimens may be used in the context of fibroblast therapeutic augmentation, and the doses may be chosen from examples of utility of anti-CD3 from the literature, as described in the following papers and incorporated by reference: prevention of kidney [14-22], liver [23-25], pancreas [26-28], lung [29], and heart [30-34] transplant rejection; prevention of graft versus host disease [35], multiple sclerosis [36], type 1 diabetes [37],

The use of monoclonal antibodies for the practice of the disclosure must be tempered by the caution that in some cases cytokine storm may be initiated by antibody administration [38, 39]. In some cases this is concentration dependent [40]. Treatment for this can be accomplished by steroid administration or anti-IL6 antibody [41-45], as certain examples.

In some embodiments of the disclosure, administration of PGE1 and/or various one or more natural anti-inflammatory compounds are provided to decrease TNF-alpha production as a result of anti-CD3 administration, such as described in this paper and incorporated by reference [46]. In further embodiments of the disclosure, administration of anti-CD3 may be performed together with endothelial protectants and/or anti-coagulants in order to reduce clotting associated with CD3 modulating agents [47]. In some embodiments, anti-CD3 antibodies may be used in combination with tolerogenic cytokines, such as interleukin-10 in order to enhance number of angiogenesis supporting T cells. The safety of anti-CD3 and IL-10 administration has previously been demonstrated in a clinical trial [48].

In the current disclosure, decreased TNF-alpha activity is correlated with enhancement of fibroblast regenerative activity. Furthermore, other inhibitors of TNF-alpha may be administered [49, 50].

In some embodiments of the disclosure, enhancement of fibroblast regenerative activity is provided by administration of one or more oral modulators of CD3. Oral administration of OKT3 has been previously performed in a clinical trial and results are incorporated by reference [51, 52].

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate butler solutions; and (21) other non-toxic compatible substances employed in pharmaceutical formulations. Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Certain embodiments of the disclosure include methods for enhancing therapeutic efficacy of a fibroblast population, comprising the steps of: a) selecting an individual in need of fibroblast therapy; b) pretreating the individual with one or more immune modulatory compounds or combination of compounds capable of augmenting therapeutic activity of the fibroblasts; c) administering the fibroblast as a therapy; d) optionally administering the immune modulatory compound or combination of compounds concurrently with and/or subsequently to administration of the fibroblasts. The fibroblast therapy may be autologous, allogeneic, or xenogeneic with respect to the individual in need of treatment.

In specific embodiments, the fibroblast therapy comprises administration of fibroblasts derived from a tissue selected from the group consisting of: a) adipose; b) omentum; c) subintestinal mucosa; d) placenta; e) cord blood; f) wharton's jelly; g) bone marrow; h) peripheral blood; i) hair follicle; j) skin; k) cutis; 1) tonsil; m) peripheral blood; n) menstrual blood; o) thymus; and p) a combination thereof. The fibroblasts may be plastic adherent, in specific embodiments. In particular cases, the fibroblasts express one or more markers selected from the group consisting of a) CD73; b) CD56; c) CD140; d) CD105; e) CD90; and f) a combination thereof.

In particular embodiments, the fibroblasts possess certain abilities, such as the ability to induce angiogenesis; inhibit apoptosis; induce activation of endogenous progenitor cells; to induce regeneration of injured tissue; to inhibit fibrosis of injured tissue; or a combination thereof. In certain embodiments, the fibroblasts possess a younger biological age as compared to the recipient. In specific embodiments, a younger biological age is induced by transfection with one or more agents capable of repairing and/or restoring telomere length, such as the fibroblasts being transfected with hTERT in order to reduce biological age. In certain cases, the fibroblasts are dedifferentiated in order to reduce biological age, and the dedifferentiation may or may not be performed by transfection of the fibroblasts of one or more factors selected from the group consisting of: a) OCT-4; b) NANOG; c) SOX-2; and d) a combination thereof. In particular cases, dedifferentiation is induced by addition of one or more agents selected from the group consisting of a) a DNA methyltransferase inhibitor; b) a histone deacetylase inhibitor; c) an inhibitor of GSK-3; and d) a combination thereof. Dedifferentiation may be accomplished by transfection of cytoplasm from a cell younger than the fibroblast. A younger cell may be a pluripotent stem cell, for example, such as one derived from somatic cell nuclear transfer, derived from the process of parthenogenesis, or generated as an inducible pluripotent stem cell.

In embodiments wherein one administers an immune modulatory compound or combination of compounds concurrently with and/or subsequently to administration of the fibroblasts, the compound may be capable of inducing generation and/or increasing numbers of T cells with immune suppressive activity. The immune suppressive activity may be an ability to inhibit mixed lymphocyte reaction, associated with enhanced angiogenesis activity (such as associated with an enhanced ability to stimulate growth factor production from the fibroblasts), and/or associated with enhanced survival of administered fibroblasts into a recipient. As specific embodiments, the immune modulatory compound may be interleukin-2 or an entity capable of activating interleukin-2 receptors, for example to induce an augmentation of T cell immune suppressive activity. In a specific case, an activator of interleukin-2 receptor is capable of inducing proliferation of CD4 T cells capable of stimulating enhancement of T cell mediated angiogenesis. In specific embodiments, interleukin-2 is administered in the form of aldesleukin. In specific cases, aldesleukin is administered every day at concentrations of 0.3×10⁶ to 3.0×10⁶ IU IL-2 per square meter of body surface area for 1-16 weeks.

In one embodiment, the immune modulatory compound is anti-CD3 antibody, and in specific cases, the anti-CD3 antibody does not possess an ability to bind fragment crystalline receptor. In specific embodiments, the anti-CD3 antibody is Teplizumab.

Kits

Any of the compositions described herein may be comprised in a kit. In a non-limiting example, fibroblasts, IL-2 and/or one or more anti-CD3 agents may be comprised in a kit. The kits will thus comprise in suitable container means the fibroblasts, IL-2 and/or one or more anti-CD3 agents.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the fibroblasts, IL-2 and/or one or more anti-CD3 agents and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition(s). In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the formulation(s) are placed, preferably, suitably allocated. The kits may also comprise a second container means for containing a sterile, pharmaceutically acceptable buffer and/or other diluent.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle, as examples only.

### REFERENCES

1. Zhao, T. X., et al., Low-dose interleukin-2 in patients with stable ischaemic heart disease and acute coronary syndromes (LILACS): protocol and study rationale for a randomised, double-blind, placebo-controlled, phase VII clinical trial. BMJ Open, 2018. 8(9): p. e022452.
2. Jyonouchi, S., et al., Phase I trial of low-dose interleukin 2 therapy in patients with Wiskott-Aldrich syndrome. Clin Immunol, 2017. 179: p. 47-53.
3. Asano, T., et al., Phase I/IIa Study of Low Dose Subcutaneous Interleukin-2 (IL-2) for Treatment of Refractory Chronic Graft Versus Host Disease. Acta Med Okayama, 2016. 70(5): p. 429-433.
4. Kennedy-Nasser, A. A., et al., Ultra low-dose IL-2 for GVHD prophylaxis after allogeneic hematopoietic stem cell transplantation mediates expansion of regulatory T cells without diminishing antiviral and antileukemic activity. Clin Cancer Res, 2014. 20(8): p. 2215-25.
5. Mizui, M. and G. C. Tsokos, Low-Dose IL-2 in the Treatment of Lupus. Curr Rheumatol Rep, 2016. 18(11): p. 68.
6. Todd, J. A., et al., Regulatory T Cell Responses in Participants with Type 1 Diabetes after a Single Dose of Interleukin-2: A Non-Randomised, Open Label, Adaptive Dose-Finding Trial. PLoS Med, 2016. 13(10): p. e1002139.
7. Pham, M. N., M. G. von Herrath, and J. L. Vela, Antigen-Specific Regulatory T Cells and Low Dose of IL-2 in Treatment of Type 1 Diabetes. Front Immunol, 2015. 6: p. 651.
8. Waldron-Lynch, F., et al., Rationale and study design of the Adaptive study of IL-2 dose on regulatory T cells in type 1 diabetes (DILT1D): a non-randomised, open label, adaptive dose finding trial. BMJ Open, 2014. 4(6): p. e005559.
9. Hirsch, R., J. D. Ellenhorn, and J. A. Bluestone, In vivo administration of anti-CD3 monoclonal antibody can activate immune responses thus preventing malignant tumor growth. Princess Takamatsu Symp, 1988. 19: p. 237-43.
10. Schoof, D. D., et al., Activation of human tumor-infiltrating lymphocytes by monoclonal antibodies directed to the CD3 complex. Cancer Res, 1990. 50(4): p. 1138-43.
11. Davis, L. S., M. C. Wacholtz, and P. E. Lipsky, The induction of T cell unresponsiveness by rapidly modulating CD3. J Immunol, 1989. 142(4): p. 1084-94.
12. Anasetti, C., et al., Induction of specific nonresponsiveness in unprimed human T cells by anti-CD3 antibody and alloantigen. J Exp Med, 1990. 172(6): p. 1691-700.
13. Woodle, E. S., et al., Phase I trial of a humanized, Fc receptor nonbinding OKT3 antibody, huOKT3gamma1(Ala-Ala) in the treatment of acute renal allograft rejection. Transplantation, 1999. 68(5): p. 608-16.
14. Cosimi, A. B., et al., Treatment of acute renal allograft rejection with OKT3 monoclonal antibody. Transplantation, 1981. 32(6): p. 535-9.
15. Ortho Multicenter Transplant Study, G., A randomized clinical trial of OKT3 monoclonal antibody for acute rejection of cadaveric renal transplants. N Engl J Med, 1985. 313(6): p. 337-42.
16. Debure, A., et al., One-month prophylactic use of OKT3 in cadaver kidney transplant recipients. Transplantation, 1988. 45(3): p. 546-53.
17. Oh, H. K., et al., Two low-dose OKT3 induction regimens following renal transplantation—clinical experience at a single center. Clin Transplant, 1998. 12(4): p. 343-7.
18. Opelz, G., Efficacy of rejection prophylaxis with OKT3 in renal transplantation. Collaborative Transplant Study. Transplantation, 1995. 60(11): p. 1220-4.
19. Darby, C. R., et al., Reduced dose OKT3 prophylaxis in sensitised kidney recipients. Transpl Int, 1996. 9(6): p. 565-9.
20. Ciancio, G., et al., Human donor bone marrow cells can enhance hyporeactivity in renal transplantation using maintenance FK 506 and OKT3 induction therapy. Transplant Proc, 1996. 28(2): p. 943-4.
21. Waid, T. H., et al., Treatment of renal allograft rejection with T10B9.1A31 or OKT3: final analysis of a phase II clinical trial. Transplantation, 1997. 64(2): p. 274-81.
22. Kumar, M. S., et al., ATGAM versus OKT3 induction therapy in cadaveric kidney transplantation: patient and graft survival, CD3 subset, infection, and cost analysis. Transplant Proc, 1998. 30(4): p. 1351-2.
23. Cosimi, A. B., et al., A randomized clinical trial comparing OKT3 and steroids for treatment of hepatic allograft rejection. Transplantation, 1987. 43(1): p. 91-5.
24. Millis, J. M., et al., Randomized prospective trial of OKT3 for early prophylaxis of rejection after liver transplantation. Transplantation, 1989. 47(1): p. 82-8.
25. Whiting, J. F., et al., Use of low-dose OKT3 as induction therapy in liver transplantation. Transplantation, 1998. 65(4): p. 577-80.
26. Melzer, J. S., et al., The use of OKT3 in combined pancreas-kidney allotransplantation. Transplant Proc, 1990. 22(2): p. 634-5.
27. Sindhi, R., et al., Increased risk of pulmonary edema in diabetic patients undergoing preemptive pancreas transplantation with OKT3 induction. Transplant Proc, 1995. 27(6): p. 3016-7.
28. Stratta, R. J., et al., A prospective randomized trial of OKT3 vs ATGAM induction therapy in pancreas transplant recipients. Transplant Proc, 1996. 28(2): p. 917-8.
29. Ross, D. J., et al., Delayed development of obliterative bronchiolitis syndrome with OKT3 after unilateral lung transplantation. A plea for multicenter immunosuppressive trials. Chest, 1996. 109(4): p. 870-3.
30. van Gelder, T., et al., A randomized trial comparing safety and efficacy of OKT3 and a monoclonal anti-interleukin-2 receptor antibody (BT563) in the prevention of acute rejection after heart transplantation. Transplantation, 1996. 62(1): p. 51-5.
31. Delgado, J. F., et al., Induction treatment with monoclonal antibodies for heart transplantation. Transplant Rev (Orlando), 2011. 25(1): p. 21-6.
32. Kormos, R. L., et al., Monoclonal versus polyclonal antibody therapy for prophylaxis against rejection after heart transplantation. J Heart Transplant, 1990. 9(1): p. 1-9, discussion 9-10.
33. Rabinov, M., et al., Recipient selection algorithm for immunosuppression in cardiac transplantation: OKT3 vs triple therapy alone. Transplant Proc, 1992. 24(1): p. 167-8.
34. Chin, C., et al., Induction therapy for pediatric and adult heart transplantation: comparison between OKT3 and daclizumab. Transplantation, 2005. 80(4): p. 477-81.
35. Prentice, H. G., et al., Use of anti-T-cell monoclonal antibody OKT3 to prevent acute graft-versus-host disease in allogeneic bone-marrow transplantation for acute leukaemia. Lancet, 1982. 1(8274): p. 700-3.
36. Weinshenker, B. G., et al., An open trial of OKT3 in patients with multiple sclerosis. Neurology, 1991. 41(7): p. 1047-52.
37. Herold, K. C., et al., A single course of anti-CD3 monoclonal antibody hOKT3gamma1(Ala-Ala) results in improvement in C-peptide responses and clinical parameters for at least 2 years after onset of type 1 diabetes. Diabetes, 2005. 54(6): p. 1763-9.
38. Ferran, C., et al., Cytokine-related syndrome following injection of anti-CD3 monoclonal antibody: further evidence for transient in vivo T cell activation. Eur J Immunol, 1990. 20(3): p. 509-15.
39. Vasquez, E. M., A. J. Fabrega, and R. Pollak, OKT3-induced cytokine-release syndrome: occurrence beyond the second dose and association with rejection severity. Transplant Proc, 1995. 27(1): p. 873-4.
40. Norman, D. J., J. A. Kimball, and J. M. Barry, Cytokine-release syndrome: differences between high and low doses of OKT3. Transplant Proc, 1993. 25(2 Suppl 1): p. 35-8.
41. Goldman, M., et al., OKT3-induced cytokine release attenuation by high-dose methylprednisolone. Lancet, 1989. 2(8666): p. 802-3.
42. Fletcher, E. A. K., et al., Extracorporeal human whole blood in motion, as a tool to predict first-infusion reactions and mechanism-of-action of immunotherapeutics. Int Immunopharmacol, 2018. 54: p. 1-11.
43. Chatenoud, L., et al., In vivo cell activation following OKT3 administration. Systemic cytokine release and modulation by corticosteroids. Transplantation, 1990. 49(4): p. 697-702.
44. Chatenoud, L., et al., Corticosteroid inhibition of the OKT3-induced cytokine-related syndrome—dosage and kinetics prerequisites. Transplantation, 1991. 51(2): p. 334-8.
45. Bugelski, P. J., et al., Monoclonal antibody-induced cytokine-release syndrome. Expert Rev Clin Immunol, 2009. 5(5): p. 499-521.
46. Barel, D., et al., Enhanced tumor necrosis factor in anti-CD3 antibody stimulated diabetic NOD mice: modulation by PGE1 and dietary lipid. Autoimmunity, 1992. 13(2): p. 141-9.
47. Pradier, O., et al., Procoagulant effect of the OKT3 monoclonal antibody: involvement of tumor necrosis factor. Kidney Int, 1992. 42(5): p. 1124-9.
48. Wissing, K. M., et al., A pilot trial of recombinant human interleukin-10 in kidney transplant recipients receiving OKT3 induction therapy. Transplantation, 1997. 64(7): p. 999-1006.
49. Charpentier, B., et al., Evidence that antihuman tumor necrosis factor monoclonal antibody prevents OKT3-induced acute syndrome. Transplantation, 1992. 54(6): p. 997-1002.
50. DeVault, G. A., Jr., et al., The effects of oral pentoxifylline on the cytokine release syndrome during inductive OKT3. Transplantation, 1994. 57(4): p. 532-40.
51. Ilan, Y., et al., Oral administration of OKT3 monoclonal antibody to human subjects induces a dose-dependent immunologic effect in T cells and dendritic cells. J Clin Immunol, 2010. 30(1): p. 167-77.
52. Lalazar, G., et al., Oral Administration of OKT3 MAb to Patients with NASH, Promotes Regulatory T-cell Induction, and Alleviates Insulin Resistance: Results of a Phase IIa Blinded Placebo-Controlled Trial. J Clin Immunol, 2015. 35(4): p. 399-407.
U.S. Pat. No. 4,530,787
U.S. Pat. No. 4,569,790
U.S. Pat. No. 4,572,798
U.S. Pat. No. 4,604,377
U.S. Pat. No. 4,748,234
U.S. Pat. No. 4,853,332
U.S. Pat. No. 4,959,314
U.S. Pat. No. 5,229,109
U.S. Pat. No. 5,464,939
U.S. Pat. No. 7,514,073
U.S. Pat. No. 7,569,215

The scope of the present application is not intended to be limited to the particular embodiments of the process, machine, manufacture, composition of matter, means, methods and steps described in the specification.

1. A method of providing a fibroblast therapy to an individual in need thereof, comprising the step of providing to the individual an effective amount of the fibroblast therapy and one or both of interleukin (IL)-2 and one or more anti-CD3 agents.

2. The method of claim 1, wherein the fibroblast therapy is autologous, allogeneic, or xenogeneic with respect to the individual.

3. The method of claim 1, wherein the fibroblasts are plastic-adherent.

4. The method of claim 1, wherein the fibroblasts are derived from a tissue selected from the group consisting of a) adipose; b) omentum; c) subintestinal mucosa; d) placenta; e) cord blood; f) Wharton's jelly; g) bone marrow; h) peripheral blood; i) hair follicle; j) skin; k) cutis; l) tonsil; m) peripheral blood; n) menstrual blood; o) thymus; and p) a combination thereof.

5. The method of claim 1, wherein the fibroblasts express one or more markers selected from the group consisting of a) CD73; b) CD56; c) CD140; d) CD105; e) CD90; and f) a combination thereof.

6. The method of claim 1, wherein the fibroblasts possess a younger biological age than the individual.

7. The method of claim 1, wherein the fibroblasts are transfected with hTERT.

8. The method of claim 1, wherein the fibroblasts are dedifferentiated.

9. The method of claim 8, wherein the fibroblasts are transfected with Oct-4, NANOG, SOX-2, or a combination thereof.

10. The method of claim 8, wherein the fibroblasts are exposed to, transfected with, or both of one or more DNA methyltransferase inhibitors, one or more histone deacetylase inhibitors, one or more inhibitors of GSK-3, or a combination thereof.

11. The method of claim 8, wherein the fibroblasts are transfected with cytoplasm from cells younger than the fibroblasts.

12. The method of claim 11, wherein the cells younger than the fibroblasts are pluripotent stem cells.

13. The method of claim 1, wherein the IL-2 is administered to the individual at a concentration of 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0×10⁶ IU/m²/day.

14. The method of claim 1, wherein the IL-2 is administered to the individual at a dose between 0.3×10⁶ IU/m²/day and 3.0×10⁶ IU/m²/day.

15. The method of claim 1, wherein the IL-2 is administered to the individual at a dose no more than 20.0×10⁶ IU/m²/day.

16. The method of claim 1, wherein the IL-2 is administered to the individual daily.

17. The method of claim 1, wherein the IL-2 is administered to the individual for 1-16 weeks.

18. The method of claim 1, wherein the IL-2 is administered daily to the individual for 1-16 weeks.

19. The method of claim 1, wherein the IL-2 is administered as a continuous infusion.

20. The method of claim 1, wherein the IL-2 is administered by subcutaneous injection.

21. The method of claim 1, wherein the IL-2 is administered prior to the fibroblasts.

22. The method of claim 21, wherein the IL-2 is administered prior to the fibroblasts in a range of about 1 hour prior to about 3 weeks prior.

23. The method of claim 22, wherein the IL-2 is administered about 1 week prior to the fibroblasts.

24. The method of claim 1, wherein the anti-CD3 agent is an antibody, aptamer, siRNA or shRNA, or combination thereof.

25. The method of claim 24, wherein the anti-CD3 agent is an antibody.

26. The method of claim 25, wherein the antibody is a monoclonal antibody.

27. The method of claim 1, wherein the individual is provided the anti-CD3 agent prior to delivery of the fibroblasts.

28. The method of claim 27, wherein the anti-CD3 agent is provided to the individual 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, and/or 1 day before administration of the fibroblasts to the individual.

29. The method of claim 1, wherein the individual is provided an effective amount of one or more tolerance inducing agents.

30. The method of claim 29, wherein the tolerance inducing agent is alpha1-antitrypsin.
